(19) United States
(12) Patent Application Publication
(10) Pub. No.: US 2022/0003741 A1
(43) Pub. Date: Jan. 6, 2022

(54) ORAL WASH FOR ORAL FLUID COLLECTION AND ANALYSIS AND METHODS OF USE THEREOF

(71) Applicant: Decimadx, LLC, Atlanta, GA (US)

(72) Inventor: Phillip Gibbs, Atlanta, GA (US)

(21) Appl. No.: 17/294,196

(22) PCT Filed: Nov. 18, 2019
(86) PCT No.: PCT/US2019/062010

§ 371 Date: May 14, 2021

Related U.S. Application Data
(60) Provisional application No. 62/768,501, filed on Nov. 16, 2018.

(51) Int. Cl. G01N 33/487; G01N 33/58

(52) U.S. Cl. CPC G01N 33/487; G01N 33/58

(57) ABSTRACT

Disclosed are oral wash compositions for collection of analyte in oral fluid sample. The oral wash includes an internal tracer, which can be directly detected by a mass-spec-based analytical method such as LC-MS, GC-MS, CE-MS, HPLC-MS/MS, HPLC-TOF or MALDI-TOF assay, along with all the other analytes being tested in the collected oral fluid. The oral wash optionally includes agents to induce salivation, maintain oral wash integrity at room temperature, and stabilize the collected oral fluid, and one or more dyes. The oral wash is used to collect saliva from the oral cavity, in order to determine the presence of an analyte in the saliva. The method includes rinsing the oral cavity with the oral wash for a period of time effective to stimulate saliva production, collecting the resulting fluid and subjecting the resulting fluid to a mass-spec-based analytical method to detect the presence of an analyte in the sample.

### CROSS-REFERENCE TO RELATED APPLICATIONS

This application claims the benefit of and priority to U.S. Provisional Application No. 62/768,501 filed Nov. 16, 2018, which is hereby incorporated by reference in its entirety.

### FIELD OF THE INVENTION

The invention is generally directed to an oral wash for oral fluid collection for subsequent analyte detection using a mass-spec-based analytical method such as HPLC-MS/MS or HPLC-TOF.

### BACKGROUND OF THE INVENTION

Saliva is a sample material that is easy to collect without causing stress and can be used for a whole series of analytical purposes. Amongst other things, saliva tests provide valuable information for diagnostic purposes and for controlling the treatment of a number of diseases. It allows proteins, hormones, metabolic metabolites, electrolytes and various pharmaceutical substances to be tested.

Collection methods used for gathering oral fluids used in diagnostic testing has profound implications for the rate of false negatives and the amount of sample clean up required for analysis. Several solutions are available (see for example, U.S. Pat. No. 7,883,724, Quantisal™) but these products are not optimal for ease of collection (too cumbersome/slow) and/or require considerable pre-analytical processing when utilized with HPLC-MS to remove dyes/surfactants. In addition, an indicator dye to tell when enough oral fluid has been collected leads to a high false negative rate since once the collector is dropped into the collection buffer, the indicator dye cannot distinguish properly collected samples from invalid ones. Another issue with oral fluid collectors from stimulated saliva production, is a way to normalize the dilution of the collected oral fluid with the oral wash/rinse. Previous methods utilize an internal tracer dye to make a photometric measurement, and subsequent calculation to determine the dilution correction factor (See U.S. Pat. No. 7,883,724). However, this approach is not ideal from the standard work flow in a method utilizing high performance liquid chromatography-tandem mass spectrometry (HPLC-MS/MS), HPLC-TOF or MALDI-TOF for analyzing drug/metabolites in oral fluids since it requires a separate measurement to determine the dilution factor.

There remains a need for oral wash compositions, which simplify sample collection from the oral cavity and the subsequent identification of analyte, using instrumentation such as LC-MS, GC-MS, CE-MS, HPLC-MS/MS, HPLC-TOF or MALDI-TOF.

Therefore, it is the object of the present invention to provide an oral wash composition, which includes a tracer detectable by LC-MS, GC-MS, CE-MS, HPLC-MS/MS, HPLC-TOF or MALDI-TOF.

It is also an object of the present invention to provide a method of detecting the presence of an analyte in a sample from the oral cavity using a mass-spec-based analytical method such as LC-MS, GC-MS, CE-MS, HPLC-MS/MS, HPLC-TOF or MALDI-TOF.

### SUMMARY OF THE INVENTION

Disclosed are oral wash compositions for collection of analyte in oral fluid sample. The oral wash includes an internal tracer, which can be directly detected by a mass-spec-based analytical method such as LC-MS, GC-MS, CE-MS, HPLC-MS/MS, HPLC-TOF or MALDI-TOF assay, along with all the other analytes being tested in the collected oral fluid. Alternatively, the oral wash does not include any internal tracer.

In a preferred embodiment, the internal tracer is a stable labelled heavy isotope. In a more preferred embodiment, the internal tracer is caffeine-¹³C₃. Optionally, the internal tracer is a stable heavy isotope-labelled variant of the analyte of interest. The disclosed oral wash composition preferably does not include tartrazine. The oral wash optionally include agents to induce salivation, maintain oral wash integrity at room temperature (prevent bacterial/fungal growth), and stabilize the collected oral fluid, and one or more dyes. The disclosed oral wash are used to detect the presence of an analyte in saliva. The oral wash composition is used to collect saliva from the oral cavity, which is subsequently subjected to a mass-spec-based analytical method such as LC-MS, GC-MS, CE-MS, HPLC-MS/MS, HPLC-TOF or MALDI-TOF, in order to determine the presence of an analyte in the saliva. The method includes rinsing the oral cavity with the disclosed oral wash for a period of time effective to stimulate saliva production, collecting the resulting fluid (oral wash plus saliva) into a container and subjecting the resulting fluid to a mass-spec-based analytical method such as LC-MS, GC-MS, CE-MS, HPLC-MS/MS, HPLC-TOF or MALDI-TOF to detect the presence of an analyte in the sample.

Compared to blood-based or urine-based detection and diagnostic methods, the use of oral samples provides an easy approach for sample collection, especially when titrating the dose is important. Urine-based detection and diagnostic methods are not useful for dose titrations. The use of the oral wash is beneficial for sample collection from children. It provides the ease of collection and minimize liability concerns.

### DETAILED DESCRIPTION OF THE INVENTION

### I. Definitions

A “saliva sample” refers to samples derived from saliva from an animal that produces saliva. Saliva is a component of oral fluid produced in most animals.

A “filtered sample” refers to a saliva sample that has been processed to remove cells by separating the cell-phase and the fluid phase of saliva. A filtered sample can have more than 50%, more than 75%, more than 95%, or a 100% removal of cells. A sample is filtered to avoid mechanical rupture of cellular elements that could contribute to the detection of unwanted analytes in the cell-free phase. A filtered sample can further exclude extraneous substances, including but not limited to, food debris. Filtering the sample prior to HPLC-MS analysis is really to remove any particulate matter (food, dirt, bacteria, etc. . . . ) that may be collected in the oral fluid from the oral cavity. “Cells”, like epithelial cells from the cheeks, are probably small contributors to the components that need to filtered out. In addition, removal is mainly to prevent these large particulates clogging up the HPLC as opposed to directly interfering with the mass spec analysis since the HPLC column+guard would filter them out anyway.

### II. Compositions

The disclosed oral wash includes an internal tracer, which can be directly detected by a mass-spec-based technique such as LC-MS, GC-MS, CE-MS, HPLC-MS/MS, HPLC-TOF or MALDI-TOF assay, along with all the other analytes being tested in the collected oral fluid. The disclosed oral wash eliminates the need for additional instrumentation other than the mass-spec-based technique to detect an internal standard in the oral wash. For example, oral washes that use an internal tracer dye which relies on a photometric measurement, and subsequent calculation, for example, to determine the dilution correction factor (U.S. Pat. No. 7,883,724). Because the internal tracer included in the disclosed oral wash can be detected by the same instrument used to detect the analyte of interest in the oral sample collected and at the same time as the analyte of interest, using the disclosed oral wash, additional instrumentation and separate sample prep is not needed.

Optionally, the pH range of the oral wash is between about 3.0 and about 6.2. The pH of the oral wash can be maintained via adding a buffering agent such as citrate. In some embodiments, the pH of the oral wash is adjusted to at or below 4.6 to improve shelf life (inhibit bacterial growth) and encourage salvation. Having an acidic pH of the oral wash can also ionize the analytes in the form of weak bases, thereby increasing total oral fluid concentrations of the analytes.

a. Internal Tacer

In a preferred embodiment, the internal tracer is a stable labelled heavy isotope. The internal tracer can facilitate dilution correction (normalization), validity conformation (preventing false negative results), and/or mitigate adsorptive losses.

Advantages of using a stable heavy isotope-labelled internal tracer is that such internal tracers cannot contribute/interfere with the signal at the most abundant monoisotopic mass. Preferred tracers are stable labelled heavy isotopes of commercially available analytes that can easily be detected and distinguished from potential background interferants.

Stable labelled isotopes are preferable over deuterated analytes due to problems of hydrogen exchange upon long-term storage in the oral wash solution (Davison, et al., Annals of Clin Biochem., 560:274 (2013)). C¹³ and N¹⁵ stable labelled isotopes are most preferred, and are commercially available.

In a more preferred embodiment, the internal tracer is heavy-isotope labeled caffeine, such as caffeine-¹³C, caffeine-¹³C₃, and caffeine-¹³C,D₃. Caffeine is ubiquitous in the population, works well with the HPLC-MS method (sharp peaks, great S/N), and has many options for heavy labelled isotopes commercially available.

Caffeine-¹³C₃ was found to be a readily available heavy labelled isotope of a common analyte that presents no danger when included in the oral wash at low concentrations (100 ng/ml) for example, between 100 and 1000 ng/ml. In addition, having the three C13's places the analyte sufficiently far enough away (+3 Da) from the native analyte as to avoid background interference from endogenous caffeine that can be present in high levels for frequent caffeine users. The presence of a background can be addressed by quantitatively measuring endogenous caffeine collected in the oral fluid and applying a correction to the caffeine-¹³C₃ value used to determine the dilution factor correction. Caffeine-¹³C,D₃- can also provide a large shift in the m/z to distinguish from endogenous caffeine. It can be added to the oral wash at the same concentration range as that of caffeine-¹³C₃.

Caffeine also has stable labelled plus deuterated versions of the analyte that allow post collection addition of an internal standard to correct both measurements caffeine and caffeine-¹³C₃ for ion enhancement/suppression due to matrix effects. In some embodiments, the internal standard is the [13C, 2H3]-caffeine, caffeine-13C-D3 deuterated standard, and/or single C13 stable labelled caffeine. This is separate and distinguishable from the stable labeledcaffeine-¹³C₃.

In some embodiments, the internal tracer is a stable labelled heavy isotope of the analyte of interest. For example, when the analyte is a small-molecule drug such as THC, methadone, EDDP, aripiprazole and metabolite (e.g., dehydro-aripiprazole or OPC-3373), the internal tracer can be a heavy isotope-labeled variant of the analyte. The heave isotope labeling can be one or more of the carbon and/or nitrogen atoms of the analyte, and/or on one or more of the hydrogen atoms of the analyte, as described above.

Optionally, the oral wash does not contain any internal tracer. For example, analytes with a high diffusion constant in water, such as alcohol, can quickly reach distribution equilibrium after the oral wash is in contact with the oral cavity. Such analytes may not need the use of any internal tracer in the oral wash for dilution correction or validity confirmation.

b. Salivation Agents

The oral wash optionally include agents to induce salivation. Examples include, but are not limited to inorganic and/or organic edible acids and/or salts or mixtures thereof, for example, phosphoric acid, lactic acid, citric acid, and ascorbic acid. Examples of salivary stimulants are disclosed for example in U.S. Pat. No. 4,820,506. Compositions useful within the context of xerostomia may also be used. For example, nutritionally acceptable and chemically defined compounds may be administered as described in U.S. Pat. App. No. 2007/0128284 where a sulfur-containing antioxidant such as N-acetylcysteine is combined with a polymeric base, or in U.S. Pat. App. No. 2004/0076695 where omega-3 fatty acids are used, in various compositions. In yet further known compositions, peroxidized lipids (typically plant oils) and silica are used to alleviate xerostomia as taught in U.S. Pat. App. No. 2006/0078620. In yet other known methods, glycerol may be employed to improve dry mouth conditions as noted in U.S. Pat. App. No. 2009/0263467. U.S. Pat. No. 9,5972,287 discloses compositions, which employ one or more plant pulp products and/or a proanthocyanidin to stimulate saliva and reduce dry mouth.

The concentration of the substance with saliva stimulating properties is selected so as to be between 0.0005% and 10% of the oral wash composition, preferably between 0.01%, and 5% and more preferably, between 1%, and 2%.

c. Active Agents

The compositions include one or more active agents to maintain oral wash integrity at room temperature, preferably, agents, which prevent bacterial/fungal growth, and/or agents, which stabilize the collected oral fluid. Examples include, but are not limited to, food-grade conservatives such as potassium benzoate. Saliva contains several bacterial proteases, which can degrade salivary proteins affecting some techniques. To avoid this pre-analytical issue, it is useful to include protease inhibitors and stabilizing substances (such as aprotinin, leupeptin, antipain, pepstatin A, phenyl methyl sulfonyl fluoride, EDTA, thimerosal).

d. Additional Components

One additional component of the oral wash that is included for aesthetics, are FDA approved food dyes (these are also synthetic dyes and essentially homogenous, one component, in contrast to natural sources which are less ideal due to heterogenous nature of the mixes). In this case, Allura Red AC was found to be a readily available dye that could be incorporated in the wash and detected on the mass-spec-based method used to detect analytes of interest. In some preferred embodiments, the compositions does not include Allura Red AC as an internal tracer.

Other dyes that can be used in the oral wash composition for aesthetic reasons include, but are not limited to, riboflavin, riboflavin-5′-phosphate, chlorophyls and chlorophyllines, copper-containing complexes of chlorophyls and Chlorophyllines, caramel dye, simple sugar-based dye, sulphite lye-caramel die, ammonia-caramel dye, ammonium sulphite-caramel die, vegetable carbons, paprika extract, capsanthin, capsorubin, beetroot, betanine, antho-cyans, iron oxides and hydroxides, azorubin, carmoisin, Ponceau 4R, cochineal red A, allura red AC, patent blue V, indigotin, indigo carmine, brilliant blue FCF, green S, brilliant black BN, black PN, brown HT, lycopene, beta-apo-8′-carotinal (C30), beta-apo-8′-carotinic acid (C30)-ethyl ester, lutein, substances of Maillard compounds, tartrazine, curcumin, saffron, quinoline yellow, sunset yellow FCF, yellow orange S, cochineal, carminic acid, carmine, and carotene. In some preferred embodiments, the oral wash solution does not include a dye such as tartrazine, curcumin, saffron, quinoline yellow, sunset yellow FCF, yellow orange S, cochineal, carminic acid, carmine, or carotene.

The concentration of the dye in the disclosed oral wash can be between 0.0001% and 5% of the oral wash, preferably between 0.005% and 1%.

The oral wash can also include flavoring agents or flavor enhancers to improve the flavor of the wash. Useful flavoring agents can include a sugar and/or sugar substitute, for example, saccharose, maltose, fructose, or sweeteners such as for example saccharin, aspartame, and/or mixtures thereof. Any orally acceptable natural or synthetic flavorant can be used, including without limitation vanillin, sage, marjoram, parsley oil, spearmint oil, cinnamon oil, oil of wintergreen (methylsalicylate), peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants and the like. Also encompassed within flavorants herein are ingredients that provide fragrance and/or other sensory effect in the mouth, including cooling or warming effects. Such ingredients illustratively include menthol, menthyl acetate, menthyl lactate, camphor, eucalyptus oil, eucalyptol, anethole, eugenol, cassia, oxanone, a-irisone, propenyl guaiethol, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), menthone glycerol acetal (MGA) and the like. In certain embodiments, the composition optionally further comprises at least one sweetener, useful for example to enhance taste of the composition. Any orally acceptable natural or artificial sweetener can be used, including without limitation dextrose, sucrose, maltose, dextrin, dried invert sugar, mannose, xylose, ribose, fructose, levulose, galactose, sucralose, corn syrup (including high fructose corn syrup and corn syrup solids), partially hydrolyzed starch, hydrogenated starch hydrolysate, sorbitol, mannitol, xylitol, maltitol, isomalt, aspartame, neotame, saccharin and salts thereof, dipeptide-based intense sweeteners, cyclamates and the like. One or more sweeteners are optionally present in a total amount depending strongly on the particular sweetener(s) selected, but typically 0.01% to 15% by weight of the composition, or 0.1% to 10% by weight.

Optionally, the oral wash contains a fluorescence dye rather than a colorimetric dye. The fluorescence dye can be used as an internal tracer for dilution correction. In preferred embodiments, the fluorescence dye does not inference with mass-spec analysis of the analyte of interest in the oral sample.

Optionally, the oral wash contains one or more proteins to minimize the loss of analyte due to surface absorption during oral sample collection, transportation, storage, and/or analysis. Preferably, the proteins are from a non-human source. Exemplary proteins include, but are not limited to, ovalbumin, albumin, and lysozyme (e.g., chicken lysozyme).

Optionally, the oral wash contains one or more mass-spec compatible detergents and/or emulsifiers. Exemplary mass-spec compatible detergents and emulsifiers include, but are not limited to, sodium 3-(4-(1,1-bis(hexyloxy)ethyl)pyridinium-1-yl)propane-1-sulfonate (PPS SILENT® surfactant), INVITROSOL®, sodium 3-((1-(furan-2-yl)undecyloxy)carbonylamino)propane-1-sulfonate (PROTESEMAX® surfactant), and sodium 3-[(2-methyl-2-undecyl-1,3-dioxolan-4-yl)methoxy]-1-propanesulfonate (RAPIGEST® surfactant).

The oral wash is preferably sterilized using methods known from the prior art, for example, sterile filtration or autoclaving.

### III. Methods of Using

The disclosed oral wash is used to collect saliva from the oral cavity, which is subsequently subjected to analysis using a mass-spec-based technique such as LC-MS, GC-MS, CE-MS, HPLC-MS/MS or HPLC-TOF, in order to determine the presence of an analyte in the saliva. The disclosed method eliminates the need for additional instrumentation (other than mass spectrometry) to detect the internal tracer in the oral wash. In contrast, oral washes that rely on an internal tracer dye requires photometric measurement (using a spectrophotometer, for example) and subsequent calculation, for example, to determine the dilution correction factor. Therefore, in preferred embodiments, the disclosed method does not include the step of photometric measurement.

Analytes that can be measured in a saliva sample include, but are not limited to calcium, magnesium cortisol, alcohol, drugs and drug metabolites, biomarkers for cancer, analytes for medication monitoring, viral RNA or RNA, and microbial RNA or DNA. Additional analytes are listed in Table 1.

In some embodiments, the oral wash contains a heavy isotope-labeled variant of the analyte as an internal tracer. In some embodiments, the oral wash contains heavy isotope-labeled caffeine as an internal tracer. In some embodiments, the oral wash does not contain any internal tracer.

TABLE 1
Analytes detected in saliva

| Analyte | Examples |
|---|---|
| Hormones | |
| Steroids | cortisol, androgens, |
| | (testosterone), estriol, |
| | estrogen, progesterone, |
| | aldosterone, DHEAS |
| Antibodies | IgG, IgA, sIgA, IgM |
| Growth Factors | EGF, NGF, VEGF, IGF |
| Cytokines and Chemokines | IL-1 beta, IL-8, IL-6, MCP-1, |
| | CX3CL1, GRO-1 alpha, |
| | troponin I, TNF alpha |
| Nucleic Acids | human DNA, microbial DNA, |
| | mRNA, siRNA, micro RNA |
| | (miR-125a and miR-200a) |
| Proteins | 100's-1,000s |
| Drugs | drugs of abuse (NIDA 5), |
| | ethanol, therapeutic drugs, |
| | anticonvulsants, |
| | antipyretic/analgesics, anti- |
| | neoplastic agents, anti- |
| | bacterial agents, |
| | bronchodilators, cotinine |

The disclosed oral wash is useful to test drugs of abuse in clinical, workplace, driving under the influence of drugs (DUID), drug treatment, and criminal justice settings. The main advantages of oral fluid collection are the simplicity and noninvasiveness of sample collection, which can be easily observed, obviating the need for special restroom facilities and same-sex collectors and making adulteration more difficult. In the U.S., oral fluid (OF) testing is expanding at a rapid pace in nonregulated workplace testing, treatment, and driving under the influence of drugs (DUID) programs. In 2004, Substance Abuse and Mental Health Services Administration (SAMHSA) proposed recommended guidelines (Table 2) for mandated federal workplace OF testing.

TABLE 2
SAMHSA, DRUID, and Talloires recommended oral fluid cutoffs.^{a} (reproduced
from Bosker et al., Clin. Chem., 55(11): 1910-1931 (2009))

| | SAMHSA | SAMHSA | DRUID | Talloires |
|---|---|---|---|---|
| | screen, | confirmation, | confirmation, | confirmation, |
| Drug/analyte | μg/L | μg/L | μg/L | μg/L |
| Cannabinoid | 4 | 2^{b} | 1^{b} | 2 |
| Opiates | 40 | | | |
| Morphine | | 40 | 20 | 20 |
| Codeine | | 40 | 20 | 20 |
| 6AM | 4 | 4 | 5 | 5 |
| Methadone | | — | 20 | 20 |
| Phencyclidine | 10 | 10 | — | — |
| Amphetamines | 50 | | | |
| Amphetamine | | 50 | 25 | 20 |
| Methamphetamine | | 50^{c} | 25 | 20 |
| MDMA | 50 | 50 | 25 | 20 |
| MDA | | 50 | 25 | 20 |
| MDEA | | 50 | 25 | 20 |
| Cocaine or benzoylecgonine | 20 | 8 | 10 | 10 |
| Benzodiazepines | — | | | |
| Flunitrazepam | | — | 1 | — |
| Diazepam | | — | 5 | — |
| Alprazolam | | — | 1 | — |
| Oxazepam | | — | 5 | — |
| Nordiazepam | | — | 1 | — |
| Lorazepam | | — | 1 | — |
| Clonazepam | | — | 1 | — |

Driving under the Influence of Drugs, Alcohol and Medicines (DRUID)
^{a}See references: SAMHSA [Department of Health and Human Services, SAMHSA; DRUID
^{b}THC.
^{c}Specimen must also contain amphetamine ≥ method limit of detection.

The disclosed methods provide the advantage of using the same instrument (e.g., LC-MS, GC-MS, CE-MS, HPLC-MS/MS, HPLC-TOF or MALDI-TOF) for detecting the analyte of interest and the internal tracer. The use of an internal tracer detectable by the same instrument used to identify analytes in an oral sample solves the problem of normalizing for dilution of the collected oral fluid (internal standard normalization) but does not require a separate method to quantitate the internal tracer. In other words, quantitation of the internal tracer allows one to back-calculate the amount of oral fluid that was collected given that the starting volume of oral wash and starting concentration of the internal tracer are known.

Another aspect of the current oral wash-lab workflow combination is the use of filter vials for minimal sample prep prior to analysis. Since there are no components of the oral wash that are particularly problematic for the LC-MS, GC-MS, CE-MS, HPLC-MS/MS, HPLC-TOF or MALDI-TOF analysis (mostly salts that elute prior to analysis, go to waste via switching valves), a simple in vial filtration can be performed (Thomson filter vials) after addition of internal tracers (normally in MeOH). This greatly reduces the time and expense required for sample prep when compared to the Solid Phase Extraction or Liquid/Liquid extraction required for other methods to remove dyes and surfactants.

Saliva is produced exclusively by three large and numerous smaller saliva glands and is predominantly generated in the oral cavity. The whole fluid present in the oral cavity originates mainly from three salivary glands: parotid, submandibular and sublingual. Minor salivary glands (bucal, labial, palatal, palatoglossal, lingual) located in the oral cavity, gingival crevicular fluid with bacteria, epithelial cells, erythrocytes, leukocytes and food debris can contribute in small volume to the formation of what is designated as “oral fluid” or “whole saliva”.

The method includes rinsing the oral cavity with the disclosed oral wash for a period of time effective to stimulate saliva production, collecting the resulting fluid (oral wash plus saliva) into a container and subjecting the resulting fluid to a mass spectrometry instrumentation such as LC-MS, GC-MS, CE-MS, HPLC-MS/MS, HPLC-TOF or MALDI-TOF to detect the presence of an analyte in the sample. Cleaning mouth with water (preferably distilled) prior to the oral rinse is preferred, to eliminate residues that may hamper analyses

The volume of the oral wash composition used for rinsing the oral cavity can be between 0.5 ml and 10 ml, for example, between 1 ml and 7 ml, for example, 1 ml. 2 ml. 3 ml, 4 ml, 5 ml, 6 ml or 7 ml. The oral wash is left in the oral cavity for a period of time effective to stimulate saliva production. The time can be 20 sec-10 mins, for example, 30 sec, 45 sec, 1 min, 2 min, 3 min, 4 min, etc. It is particularly preferred to leave the oral wash in the oral cavity for a period of nor more than 1 minute.

The resultant mixture of oral wash plus saliva is then subjected to a mass spectrometry instrumentation such as LC-MS, GC-MS, CE-MS, HPLC-MS/MS, HPLC-TOF or MALDI-TOF in order to detect the analyte in the collected saliva. Saliva samples should be refrigerated at 4° C. for processing within 3 to 6 hours after collection, or no longer than 24 hours at 4° C., or the samples can be stored at −20° C. for longer.

In some preferred embodiments, the saliva sample is filtered to provide a saliva sample free of cells and removal of any “particulate matter”. Centrifugation can also be used for this purpose. The phrase “free of cells” refers to a sample solution that has been filtered in accordance with the methods of the present invention such that the sample solution is completely or substantially cell-free. Exemplary filters can include, but are not limited to, cellulose fiber matrix, hydrophilic filters, such as those based on polyvinylidene fluoride membrane, or filters based on polypropylene membrane. Filters can have micropores that are a wide variety of sizes, including, but not limited to, 0.22 μm, 0.45 μm and 5.0 μm. The term “filtering” refers to the application of a liquid sample containing cells, e.g. a saliva sample, to a membrane filter. Filtering is the process of removing cells and/or parts of cells from excess fluid in a liquid sample by passing the sample through a microporous membrane filter.

Because of the use of an internal tracer that is detectable by a mass-spec-based technique such as LC-MS, GC-MS, CE-MS, HPLC-MS/MS, HPLC-TOF or MALDI-TOF, there really isn't any extensive prep of the samples prior to analysis. The sample is loaded I the filter vial according to manufactures instructions (450 μL max, for example, 430 μL collect oral fluid+20 μL of MeOH with internal standards (typically deuterated since exchange isn't an issue in the MeOH and the time frame post dilution is too short for this to be of concern) for the HPLC-MS/MS analysis. The ratio is important to keep the prepped samples MeOH content less than the starting MeOH % for the chromatography (5% in this case, prepped sample has ˜4.5%). The top part of the filter vial is pressed in the filter the sample and this is then loaded onto the Mass spec for analysis. This is much easier and cheaper than the sample prep required from other oral collection kits like Quantisal where Solid Phase or Liquid/Liquid extraction have to be utilized to clean up and/or concentrate the sample prior to analysis. Otherwise a dilute and shoot method will “dirty” the mass spec very quickly and require frequent mass spec cleaning.

A preferred the internal tracer is caffeine-¹³C₃. In embodiments where extremely high levels of endogenous caffeine could potentially contribute to detected internal standard levels, a background correction can easily be applied by quantifying the caffeine levels in the collected oral fluids.

An additional feature of this approach to simplify the methods application in the lab has the Laboratory Information Management System (LIMS) implement the dilution calculation directly in the software that generates the analyte reports from the LC-MS, GC-MS, CE-MS, HPLC-MS/MS, HPLC-TOF or MALDI-TOF data. This algorithm can make any necessary caffeine background correction on a per sample basis, determine the undiluted internal tracer level from QCs run with the batch, and use the calculated dilution factor to correct reported concentration levels. This automates the calculation process and simplifies the workflow from the standpoint of the lab technician.

a. Cancer Diagnostics

The disclosed oral wash can be used for cancer diagnostics. Compared to conventional cancer diagnostic methods, the use of oral samples is non-invasive and/or can allow for detection of a range of cancer-related biomarkers after a one-time sample collection. In some embodiments, the method involves detection of a single cancer-related biomarker. In some embodiments, the method involves detection of a panel of cancer-related biomarkers.

In some embodiments, the oral wash contains a heavy isotope-labeled variant of the analyte as an internal tracer. In some embodiments, the oral wash contains heavy isotope-labeled caffeine as an internal tracer. In some embodiments, the oral wash does not contain any internal tracer.

Salivary biomarkers for cancer are generally known in the art. See, for example, Ngamchuea et al., Analyst, 2018, 143, 81-99; Castagnola et al., Acta Otorhinolaryngologica Italica, 2017; 37:94-101; AI-Tarawneh et al., OMICS, 2011, 15(6):353-61; and Saxena et al., Adv Biomed Res. 2017; 6: 90.

The cancer-related biomarkers include small molecules such as nitrates and nitrites, uric acid, valine, lactic acid, phenylalanine, propionylcholine, N-acetyl-L-phenylalanine, sphinganine, phytosphingosine and S-carboxymethyl-L-cysteine, or a combination thereof.

The cancer-related biomarkers also include large molecules such as proteins and nucleic acids (DNA and RNA, especially mRNA). Exemplary protein and nucleic acid-based salivary biomarkers are shown in Tables 3-5. Additional protein-based salivary biomarkers include interleukins (such as interleukins 6, 8 and 1b), cyclin D1 thioredoxin, profiling 1, resistin, thrombospondin-2, S100A8, α1-antitrypsin (AAT), haptoglobin β chains (HAP), complement C3, 4B, factor B, leucine-rich α-2-glycoprotein, galectine-7, 2-macroglobulin, ceruloplasmin, cystatin B, triose-phosphate isomerase and a protein called “deleted in malignant tumor 1 protein”, keratin 10, and haemopexin and transthyretin.

TABLE 3
The salivary tumor markers in various malignancies (reproduced
from Saxena et al., Adv Biomed Res. 2017; 6: 90)

| Malignancies | Salivary tumor markers |
|---|---|
| Breast cancer | Estrogen receptor α |
| | CA 15-3, HER2/neu, p53 |
| Ovarian cancer | CA 125 |
| Salivary gland tumors | Salivary leptin |
| Hepatocellular carcinoma | Alpha-fetoprotein |
| Pancreatic cancer | ACRV1: DMX like 2, DMXL2 |
| | and, catalytic subunit, DPM1 |
| Adenocarcinoma pancreas | CA 19-9 |

ACRV1: Acrosomal vesicle protein 1,
DPM1: Dolichyl phosphate mannosyltransferase polypeptide 1,
HER2: Human epidermal growth factor receptor 2,
CA: Cancer antigen

TABLE 4
Summary of the tumor markers in the diagnosis of oral carcinoma
(reproduced from Saxena et al., Adv Biomed Res. 2017; 6: 90)

| | Salivary | | |
|---|---|---|---|
| | transcriptome | | |
| Salivary genomic markers | markers | Salivary protein markers | Salivary microbiota |
| Somatic mutations in tumor | IL-8 | Elevated levels of | Significant increase in the |
| suppressor genes (p53) | | defensin-1 | levels of |
| | | | Porphyromonas gingivalis, |
| | | | Tannerella Forsythia |
| | | | and Candida albicans |
| Less of heterozygosity in | H3F3A | Elevated CD44 | Significantly elevated levels of |
| chromosome 3p, 9q, 13q | | | Bacteroides melaninogenica |
| and 17p | | | and Streptococcus mitis |
| Promoter hypermethylation of | IL1β | Elevated IL-8 | Presence of HPV and EBV |
| genes (p16, MGMT, or DAP-K) | | | |
| Cyclin D1 gene amplification | S100P | SCC-Ag | |
| Decrease in 3-oxoguanine DNA | DUSP1 | Calcyclin, Rho GDP | |
| glycosylase, phosphorylated-Src | | dissociation inhibitor | |
| and mammary serine protease | | | |
| inhibitor (Maspin) | | | |
| Microsatellite alterations of DNA | OAZ1 | CEA, carcinoantigen | |
| | SAT (spermidine/ | (CA19-9), CA128 | |
| | spermine | Intermediate filament | |
| | N1-acetyltransferase) | protein (Cyfra 21-1) | |
| | | RNS | |
| | | 8-OHdG DNA damage | |
| | | marker LDH) | |

H3F3A: H3 histone, family 3A,
DUSP1: Dual specificity phosphatase 1,
SCC-Ag: Squamous cell carcinoma antigen 2,
IL: Interleukin,
OAZ1: Ornithine decarboxylase antizyme 1,
CEA: Carcino-embryonic antigen,
RNS: Reactive nitrogen species,
LDH: Lactate dehydrogenase,
HPV: Human papilloma virus,
EBV: Epstein-Barr Virus,
CA: Cancer antigen

TABLE 5
Exemplary nucleic acid-based biomarkers for genetic diagnosis of oral squamous cell carcinoma
(reproduced from Markopoulos et al., The Open Dentistry Journal, 2010, 4, 172-178)

| Changes in the cellular DNA | Altered mRNA transcripts | Altered protein markers |
|---|---|---|
| Allelic loss on chromosomes 9p | Presence of IL8 | Elevated levels of defensin-1 |
| Mitochondrial DNA mutations | Presence of IL1B | Elevated CD44 |
| p53 gene mutations | DUSP1 (dual specificity phosphatase 1) | Elevated IL-6 andIL-8 |
| Promoter hypermethylation of genes (p16, | H3F3A (H3 histone, family 3A) | Inhibitor of apoptosis (IAP) |
| MGMT, or DAP-K) | | |
| Cyclin D1 gene amplification | OAZ1 (ornithine decarboxylase antizyme 1) | Squamous cell carcinoma associated |
| | | antigen (SCC-Ag) |
| Increase of Ki67 markers | S100P (S100 calcium binding protien P) | Carcino-embryonic antigen (CEA) |
| Microsatellite alterations of DNA | SAT (spermidine/sperminie N1-acetyltransferase) | Carcino-antigen (CA19-9) |
| Presence of HPV | | CA128 |
| | | Serum tumor marker (CA125) |
| | | Intermediate filament protein (Cyfra 21-1) |
| | | Tissue polypeptide specific antigen (TPS) |
| | | Reactive nitrogen species (RNS) |
| | | 8-OHdG DNA damage marker |
| | | Lactate dehydrogenase (LDH) |
| | | Immunoglobulin (IgG) |
| | | s-IgA |
| | | Insulin growth factor (IGF) |
| | | Metalloproteinases MMP-2 and MMP-11 |

The oral wash can be used to detect, diagnose or monitor different types of cancer, including but not limited to, oral cavity cancer, gastric cancer, breast cancer, ovarian cancer, salivary gland tumor, hepatocellular carcinoma, pancreatic cancer, and adenocarcinoma pancreas. In preferred embodiments, the oral wash can be used to detect, diagnose or monitor oral cavity cancer such as oral squamous cell carcinoma.

b. Detection of Drugs and Drug Metabolites

The oral wash can be used to detect drugs and drug metabolites. Compared to blood-based detection methods, the use of oral samples is non-invasive and/or can allow for quick, safe, and easy sample collection, especially for in-field applications. Compared to urine-based detection methods, the use of oral samples can provide more accurate time-related information on drug consumption.

In some embodiments, the oral wash contains a heavy isotope-labeled variant of the analyte as an internal tracer. In some embodiments, the oral wash contains heavy isotope-labeled caffeine as an internal tracer. In some embodiments, the oral wash does not contain any internal tracer.

### Opioids

Exemplary opioids that can be detected using the quantitative point-of-care assay include morphine, codeine, thebaine, heroin, hydromorphone, hydrocodone, oxycodone, oxymorphone, desomorphine, nicomorphine, propoxyphene, dipropanoylmorphine, benzylmorphine, ethylmorphine, buprenorphine, fentanyl, pethidine, meperidine, methadone, tramadol, dextropropoxyphene, or analogues or derivatives thereof. For example, oxycodone (OxyContin®) is an opioid analgesic medication synthesized from opium-derived thebaine. Percocet is a combination of oxycodone and acetaminophen (paracetamol). Vicodin is a combination of hydrocodone and acetaminophen (paracetamol). In preferred embodiments, the assay quantitatively measures oxycodone, hydrocodone, or a combination thereof.

Exemplary opioid metabolites that can be detected using the disclosed quantitative lateral flow immunoassay are shown in Table 6.

TABLE 6
Opioid metabolites

| | Key metabolizing | |
|---|---|---|
| Opioid | enzyme(s) | Major metabolites |
| Buprenorphine | CYP3A4 | Norbuprenorphine, |
| | | glucuronides |
| Codeine | CYP3A4, 2D6 | Morphine, glucuronides |
| Fentanyl | CYP3A4 | Norfentanyl |
| Hydrocodone | CYP3A4, 2D6 | Hydromorphone, |
| | | norhydrocodone |
| Hydromorphone | UGT1A3, 2B7 | Glucuronides |
| Meperidine | CYP3A4, 2B6, 2C19 | Normeperidine |
| Methadone | CYP2B6 | EDDP |
| Morphine | UGT2B7 | Glucuronides |
| Oxycodone | CYP3A4, 2D6 | Noroxycodone, oxymorphone |
| Oxymorphone | UGT2B7 | 6-OH-oxymorphone, |
| | | oxymorphone-3-glucuronide |
| Propoxyphene | CYP3A4 | Norpropoxyphene |
| Tramadol | CYP2D6 | O-desmethyl tramadol |

EDP = 2-Ethyl-1,5-dimethyl-3,3-diphenylpyrrolinium.

### Marijuana and Cannabinoids

In the cannabis plant, THC occurs mainly as tetrahydrocannabinol carboxylic acid (THC—COOH). Geranyl pyrophosphate and olivetolic acid react, catalyzed by an enzyme to produce cannabigerolic acid, which is cyclized by the enzyme THC acid synthase to give THC—COOH. Over time, or when heated, THC—COOH is decarboxylated producing THC. THC is metabolized mainly to 11-OH-THC (11-hydroxy-THC, denoted as HTHC) by the human body. This metabolite is still psychoactive and is further oxidized to 11-Nor-9-carboxy-THC (THC—COOH). More than 100 metabolites n humans and animals can be identified, but 11-OH-THC and THC—COOH are the dominating metabolites. Metabolism occurs mainly in the liver by cytochrome P450 enzymes CYP2C9, CYP2C19, and CYP3A4. More than 55% of THC is excreted in the feces and approximately 20% in the urine. The main metabolite in urine is the ester of glucuronic acid and THC—COOH and free THC—COOH. In the feces, mainly 11-OH-THC is detected.

THC, 11-OH-THC (HTHC), and THC—COOH can be detected and quantified in blood, urine, hair, oral fluid or sweat. The concentrations obtained from such analyses can often be helpful in distinguishing active from passive use or prescription from illicit use, the route of administration (oral versus smoking), elapsed time since use and extent or duration of use.

### Lithium

Lithium compounds, also known as lithium salts, are primarily used as a psychiatric medication. This includes the treatment of major depressive disorder that does not improve following the use of other antidepressants, and bipolar disorder. Lithium compounds are generally taken by mouth. Exemplary lithium compounds include, but are not limited to, lithium carbonate, lithium citrate, lithium orotate, lithium bromide, lithium chloride, lithium fluoride, and lithium iodide.

### Nicotine

As nicotine enters the body, it is distributed quickly through the bloodstream and crosses the blood-brain barrier reaching the brain within 10-20 seconds after inhalation. The elimination half-life of nicotine in the body is around two hours. The amount of nicotine absorbed by the body from smoking depends on many factors, including the types of tobacco, whether the smoke is inhaled, and whether a filter is used. For chewing tobacco, dipping tobacco, snus and snuff, which are held in the mouth between the lip and gum, or taken in the nose, the amount released into the body tends to be much greater than smoked tobacco.

Nicotine is metabolized in the liver by cytochrome P450 enzymes (mostly CYP2A6, and also by CYP2B6). A major metabolite of nicotine that is excreted in the urine is cotinine, which is a reliable and necessary indicator of nicotine usage. Other primary metabolites include nicotine N′-oxide, nornicotine, nicotine isomethonium ion, 2-hydroxynicotine and nicotine glucuronide. Glucuronidation and oxidative metabolism of nicotine to cotinine are both inhibited by menthol, an additive to mentholated cigarettes, thus increasing the half-life of nicotine in vivo.

Nicotine (cotinine) can be quantified in blood, plasma, or urine to confirm a diagnosis of poisoning or to facilitate a suspected nicotine overdose related death investigation. Urinary or salivary cotinine concentrations are frequently measured for the purposes of pre-employment and health insurance medical screening programs. Careful interpretation of results is important, since passive exposure to cigarette smoke can result in significant accumulation of nicotine, followed by the appearance of its metabolites in various body fluids.

The CYP2A6 enzyme is genetically polymorphic with certain alleles predicting altered metabolic activity. As the primary enzyme for nicotine metabolism, variation in the metabolic activity of CYP2A6 has a significant effect on an individual's level of tobacco consumption. The reduced metabolism phenotype leads to higher blood/nicotine levels and smokers tend to compensate for this by smoking less. Conversely, individuals with increased metabolic rate tend to smoke more. Lower nicotine metabolism with CYP2A6 variants also has an effect on smoking cessation, with slow metabolizers demonstrating higher levels of cessation in transdermal nicotine therapy trials. This may be due to the higher therapeutic doses of nicotine that the slow metabolizer sub-group obtains from comparable levels of transdermal nicotine treatment. Normal metabolizers have lower cessation rates probably as a result of current treatments failing to provide high enough levels of replacement blood nicotine. These normal metabolizers may be candidates for higher-dose nicotine replacement, which might potentially give rise to adverse effects in those with impaired nicotine metabolism.

The disclosed compositions and methods may be used to evaluate a patient's metabolism of nicotine. For example, nicotine levels can be quantified using the disclosed compositions and methods after a controlled dosage of nicotine is administered to a patient. This can in some embodiments involve allowing the subject to smoke a cigarette. In preferred embodiments, a nicotine patch or gum is given to the subject for a prescribed amount of time. The amount of nicotine or a metabolite thereof (e.g., cotinine) in a biological sample of the subject may then be monitored for rate of change.

### Stimulants

Stimulants (also often referred to as psychostimulants or colloquially as uppers) is an overarching term that covers many drugs including those that increase activity of the central nervous system and the body, drugs that are pleasurable and invigorating, or drugs that have sympathomimetic effects. Stimulants are widely used throughout the world as prescription medicines as well as without a prescription (either legally or illicitly) as performance-enhancing or recreational drugs.

Amphetamines are a class of stimulants based on the amphetamine structure. They include all derivative compounds which are formed by replacing, or substituting, one or more hydrogen atoms in the amphetamine core structure with substituents. Examples of substituted amphetamines includes amphetamine (itself), methamphetamine, ephedrine, cathinone, phentermine, mephentermine, bupropion, methoxyphenamine, selegiline, amfepramone, pyrovalerone, MDMA (ecstasy), and DOM (STP). Many drugs in this class work primarily by activating trace amine-associated receptor 1 (TAAR1); in turn, this causes reuptake inhibition and effluxion, or release, of dopamine, norepinephrine, and serotonin. An additional mechanism of some amphetamines is the release of vesicular stores of monoamine neurotransmitters through VMAT2, thereby increasing the concentration of these neurotransmitters in the cytosol, or intracellular fluid, of the presynaptic neuron.

Cocaine and its analogues are another class of stimulants. They usually maintain a benzyloxy connected to the carbon 3 of a tropane. Various modifications include substitutions on the benzene ring, as well as additions or substitutions in place of the normal carboxylate on the tropane 2 carbon. Various compounds with similar structure-activity relationships to cocaine that are not technically analogues have been developed as well. Exemplary cocaine analogues include stereoisomers of cocaine, 3β-phenyl ring substituted analogues, 2β-substituted analogues, N-modified analogues of cocaine, 3β-carbamoyl analogues, 3β-alkyl-3-benzyl tropanes, 6/7-substituted cocaines, 6-alkyl-3-benzyl tropanes, and piperidine homologues of cocaine. Examples of cocaine analogues can be find in Singh, Chemistry, Design, and Structure—Activity Relationship of Cocaine Antagonists, Chem. Rev., 2000, 100, 925-1024.

### Central Nerve System Depressants

Central nerve system (CNS) depressants typically slow brain activity, which makes them useful for treating anxiety and sleep problems.

Common CNS depressants include barbiturates such as pentobarbital (NEMBUTAL®), benzodiazepines, and sleep medications such as eszopiclone (LUNESTA), zaleplon (SONATA®), and zolpidem (AMBIEN®).

Benzodiazepines (BZD, BDZ, BZs), sometimes called “benzos”, are a class of psychoactive drugs whose core chemical structure is the fusion of a benzene ring and a diazepine ring. Benzodiazepines enhance the effect of the neurotransmitter gamma-aminobutyric acid (GABA) at the GABA_{A} receptor, resulting in sedative, hypnotic (sleep-inducing), anxiolytic (anti-anxiety), anticonvulsant, and muscle relaxant properties. High doses of many shorter-acting benzodiazepines may also cause anterograde amnesia and dissociation. These properties make benzodiazepines useful in treating anxiety, insomnia, agitation, seizures, muscle spasms, alcohol withdrawal and as a premedication for medical or dental procedures. Exemplary benzodiazepines include brotizolam, midazolam, triazolam, alprazolam, estazolam, flunitrazepam, clonazepam, lormetazepam, lorazepam, nitrazepam, temazepam, diazepam, clorazepate, chlordiazepoxide, flurazepam, halazepam, prazepam, oxazepam, nimetazepam, adinazolam, climazolam, loprazolam, and derivatives thereof.

### Other Drugs and Drug Metabolites

Hallucinogens are psychoactive agents, which can cause hallucinations, perceptual anomalies, and other substantial subjective changes in thoughts, emotion, and consciousness. The common types of hallucinogens are psychedelics, dissociatives, and deliriants. Although hallucinations are a common symptom of amphetamine psychosis, amphetamines are not considered hallucinogens, as they are not a primary effect of the drugs themselves. Exemplary hallucinogens include ketamine, LSD and other ergotamine derivatives, mescaline and other phenethylamines, PCP, psilocybin, salvia, DMT and other tryptamines, and ayahuasca. Psychedelics include serotonergics and cannabinoidergics. Serotonergics can be further divided into indoles/tryptamines (such as psilocybin, ergolines such as LSD, beta-carbolines, and complexly substituted tryptamines such as ibogaine) and phenethylamines such as mescaline.

γ-Hydroxybutyric acid (GHB), also known as 4-hydroxybutanoic acid, is a naturally occurring neurotransmitter and a psychoactive drug. It is a precursor to GABA, glutamate, and glycine in certain brain areas. It acts on the GHB receptor and is a weak agonist at the GABA_{B} receptor. Its prodrugs and analogs include 3-methyl-GHB, 4-methyl-GHB, 4-phenyl-GHB, 4-hydroxy-4-methylpentanoic acid (UMB68), γ-valerolactone (GVL), 1,4-butanediol diacetate (BDDA/DABD), methyl-4-acetoxybutanoate (MAB), ethyl-4-acetoxybutanoate (EAB), and γ-hydroxybutyraldehyde (GHBAL).

Additional drugs include mitragynine, 7-hydroxymitragynine, and derivatives thereof.

Additional drugs also include steroids such as nandrolone (OXANDRIN®), oxandrolone (ANADROL®), oxymetholone (ANADROL-50®), and testosterone cypionate (DEPO-TESTOSTERONE®).

Additional drugs also include Methylphenidate, ethylphenidate, and ritalinic acid. Methylphenidate is a stimulant medication used to treat attention deficit hyperactivity disorder (ADHD) and narcolepsy. Ethylphenidate acts as both a dopamine reuptake inhibitor and norepinephrine reuptake inhibitor, meaning it effectively boosts the levels of the norepinephrine and dopamine neurotransmitters in the brain, by binding to, and partially blocking the transporter proteins that normally remove those monoamines from the synaptic cleft. Ritalinic acid is a substituted phenethylamine and an inactive major metabolite of the psychostimulant drugs methylphenidate and ethylphenidate.

Aripiprazole is an atypical antipsychotic. It is primarily used in the treatment of schizophrenia and bipolar disorder. Other uses include as an add-on treatment in major depressive disorder, tic disorders and irritability associated with autism. A metabolite of aripiprazole, OPC-3373, is much more water soluble and can partition to oral fluids better than the parent compound and a more commonly assayed metabolite dehydro aripiprazole. All of the foregoing three compounds can be the analytes.

c. Alcohol Detection

When coupled with GC-MS, the oral wash can be used for alcohol detection. Compared to conventional alcohol breathometers, which may only provide presumptive results, the use of oral samples generate much more robust testing results that can be directly correlated with standard blood tests. Further, the use of oral samples can allow for in-field tests that will eliminate unnecessary time delay. In some embodiments, the oral wash contains ¹³C and/or ²H-labeled alcohol as an internal tracer. In some embodiments, the oral wash contains heavy isotope-labeled caffeine as an internal tracer. In some embodiments, the oral wash does not contain any internal tracer.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. An oral wash composition, comprising an internal tracer suitable for detection by mass spectrometry, along with one or more analytes being tested in a collected oral fluid.

2. The composition of claim 1, wherein the internal tracer contains one or more heavy isotopes.

3. The composition of claim 2, wherein the one or more heavy isotopes are selected from 13C, 15N, 2H, and combinations thereof.

4. The composition of claim 1, wherein the internal tracer is caffeine-13C3.

5. The composition of claim 1, wherein the internal tracer is a heavy-isotope labeled variant of the analyte.

6. The composition of claim 1, wherein the internal tracer is not tartrazine.

7. The composition of claim 1, further comprising one or more agents selected from the group consisting of saliva stimulating agents, active agents to prevent bacterial/fungal growth, dyes, flavor enhancing agents, and sweetening agents.

8. The composition of claim 1, wherein the composition has a pH between about 3.0 and about 6.2.

9. The composition of claim 8, wherein the composition has a pH between about 3.0 and about 4.6.

10. A method for identifying an analyte in the saliva of a subject, comprising:
(a) rinsing the oral cavity of the subject with the oral wash composition of claim 1 for a period of time effective to stimulate saliva production;
(b) collecting a resulting oral fluid from step (a) into a container, wherein the oral fluid comprises the oral wash and the saliva of the subject; and
(c) subjecting the oral fluid to a mass spectrometry instrumentation to detect the analyte.

11. The method of claim 10, wherein the analyte is a drug of abuse.

12. The method of claim 11, wherein the drug of abuse is selected from the group consisting of alcohol, cannabinoids, opioids, amphetamines, cocaine, benzodiazepines, and combinations thereof.

13. The method of claim 10, wherein the analyte is a lithium compound.

14. The method of claim 10, wherein the analyte is a biomarker for cancer.

15. The method of claim 14, wherein the biomarker is a protein.

16. The method of claim 14, wherein the biomarker is a nucleic acid.

17. The method of claim 10, wherein the mass spectrometry instrumentation is LC-MS, GC-MS, CE-MS, HPLC-MS/MS, HPLC-TOF or MALDI-TOF.

18. The method of claim 10, wherein the method does not contain any step of photometric measurement.
